(19) United States
(12) Patent Application Publication
(10) Pub. No.: US 2010/0240119 A1
(43) Pub. Date: Sep. 23, 2010

(54) MICROSEPARATION STRUCTURE AND DEVICES FORMED THEREWITH

(76) Inventor: Inlik Zainiev, West Bloomfield, MI (US)

Correspondence Address: GIFFORD, KRASS, SPRINKLE,ANDERSON & CITKOWSKI, P.C, PO BOX 7021, TROY, MI, 48007-7021, US

(21) Appl. No.: 12/708,256

(22) Filed: Feb. 18, 2010

Related U.S. Application Data
(60) Provisional application No. 61/153,443, filed on Feb. 18, 2009.

(51) Int. Cl. B01D 11/04; C12M 1/34; B01D 15/08

(52) U.S. Cl. 435/287.1; 422/255; 210/198.2

(57) ABSTRACT

A microseparation structure is provided that includes a top surface defining a first chamber. A second chamber is provided that is in fluid communication with the first chamber and characterized by a volume less than the volume of the first chamber. At least one hole extends in fluid communication with the second chamber to transmit fluids into a capillary draw void volume filled with a capillary draw-inducing agent such that liquid placed in the first chamber is drawn through the second chamber and through the hole to the capillary draw void volume filled with capillary draw-inducing agent. Particles of interest within the liquid are unable to pass into the hole and are therefore isolated within the second chamber. This structure is amenable to forming into a compact chromatographic filtration media made up of multiple such structures that is well suited for sealed testing for diseases such as malaria.

### CROSS-REFERENCE TO RELATED APPLICATIONS

This application claims priority of U.S. Provisional Patent Application Ser. No. 61/153,443 filed Feb. 18, 2009, which is incorporated herein by reference.

### FIELD OF THE INVENTION

The present invention in general relates to size separation of particles dissolved or suspended in a liquid and in particular to a separation structure having three interconnected layers of successively dimensionally decreasing cross section such that liquid filling the largest cross-sectional area layer is drawn through an inventive structure by capillary draw.

### BACKGROUND OF THE INVENTION

Separating particles from a liquid such as malformed erythrocytes from blood or finding parasites within water is labor intensive as a result of the need to pipette aliquots of the liquid and subsequent separation associated with immunochemistry, chromatography, sedimentation rates or other conventional separation techniques. These problems are compounded in instances where the particle of interest is found at low concentrations such that only a single such particle or a few such particles is likely to be found in any given aliquot. The effort and equipment typically required to perform a conventional such separation precludes field use of such separation thereby making field testing problematic.

Thus, there exists a need for a microseparation structure and devices using the same that control liquid aliquot size and perform separation without resort to a pipette or additional equipment.

### SUMMARY OF THE INVENTION

A microseparation structure is provided that includes a top surface defining a first chamber. A second chamber is provided that is in fluid communication with the first chamber and characterized by a volume less than the volume of the first chamber. At least one hole extends in fluid communication with the second chamber to transmit fluids into a capillary draw void volume filled with a capillary draw-inducing agent such that liquid placed in the first chamber is drawn through the second chamber and through the hole to the capillary draw void volume filled with capillary draw-inducing agent. Particles of interest within the liquid are unable to pass into the hole and are therefore isolated within the second chamber. This structure is amenable to forming into a compact chromatographic filtration media made up of multiple such structures that is well suited for sealed testing for diseases such as malaria.

### BRIEF DESCRIPTION OF THE DRAWINGS

FIG. 1 is a cross-sectional view of an inventive microseparation structure;

FIGS. 2.1-2.5 are cross-sectional views of the assembly of an inventive microseparation device;

FIGS. 3.1-3.4 are paired cross-sectional and perspective views of the operation of the device of FIG. 2.5;

FIG. 4 is a set of corresponding exploded view (FIG. 4.1), perspective view (FIG. 4.2), and cross-sectional views (FIG. 4.3) of an assembly of another embodiment of separation device;

FIGS. 5.1-5.6 are cross-sectional views of the operative steps in performing a microseparation with the device of FIG. 4;

FIGS. 6.1 and 6.2 are paired cross-sectional and top views of detection of particles in a common first chamber device of FIG. 4 for malaria detection;

FIGS. 7.1 and 7.2 are paired cross-sectional and top views of detection of particles in a multi-well first chamber device similar to that of FIG. 4 for parallel testing of multiple samples;

FIG. 8 is a set of corresponding exploded view (FIG. 8.1), perspective view (FIG. 8.2), and cross-sectional views (FIG. 8.3) of an assembly of another embodiment of separation device;

FIGS. 9.1-9.4 are cross-sectional views of the operative steps in performing a microseparation with the device of FIG. 8;

FIG. 10.1 is a top view of another embodiment of an inventive device containing a membrane in lieu of holes;

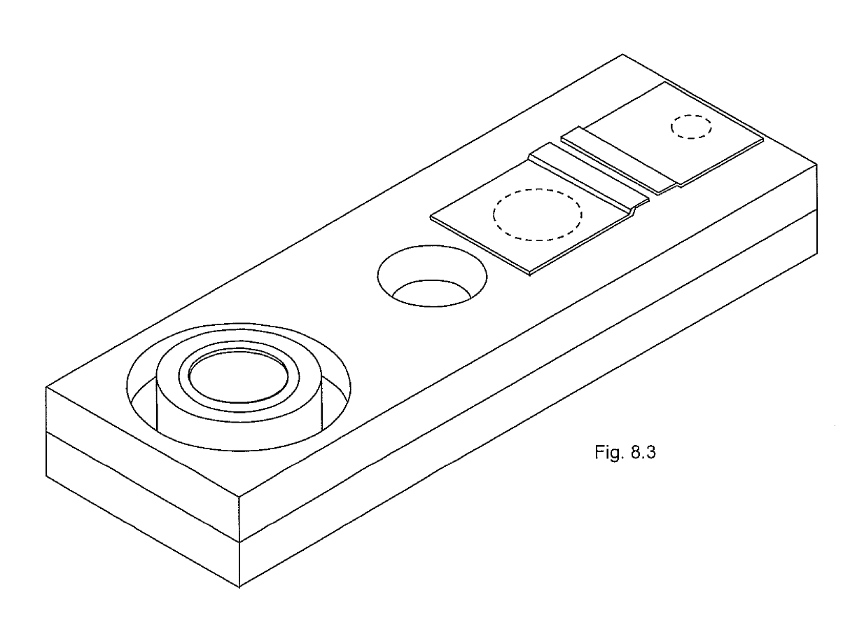

FIG. 10.2 is a cross-sectional magnified view of the membrane in FIG. 10.1; and

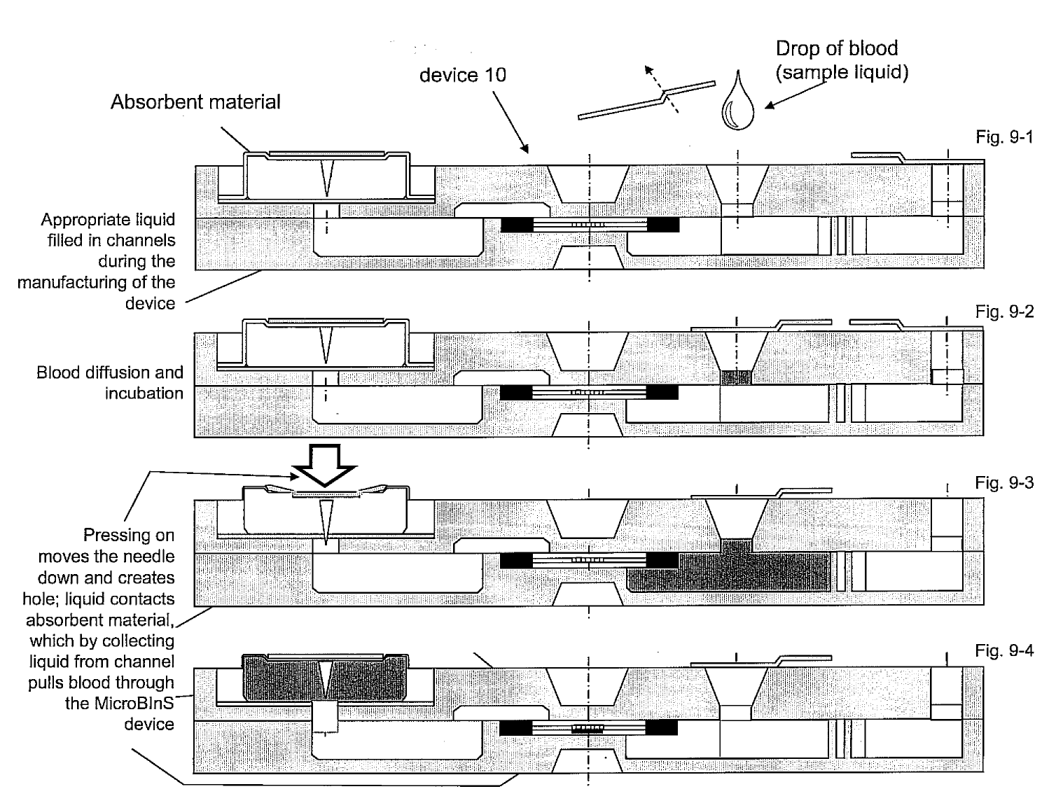

FIG. 10.3 is a perspective view of the device of FIG. 10.1.

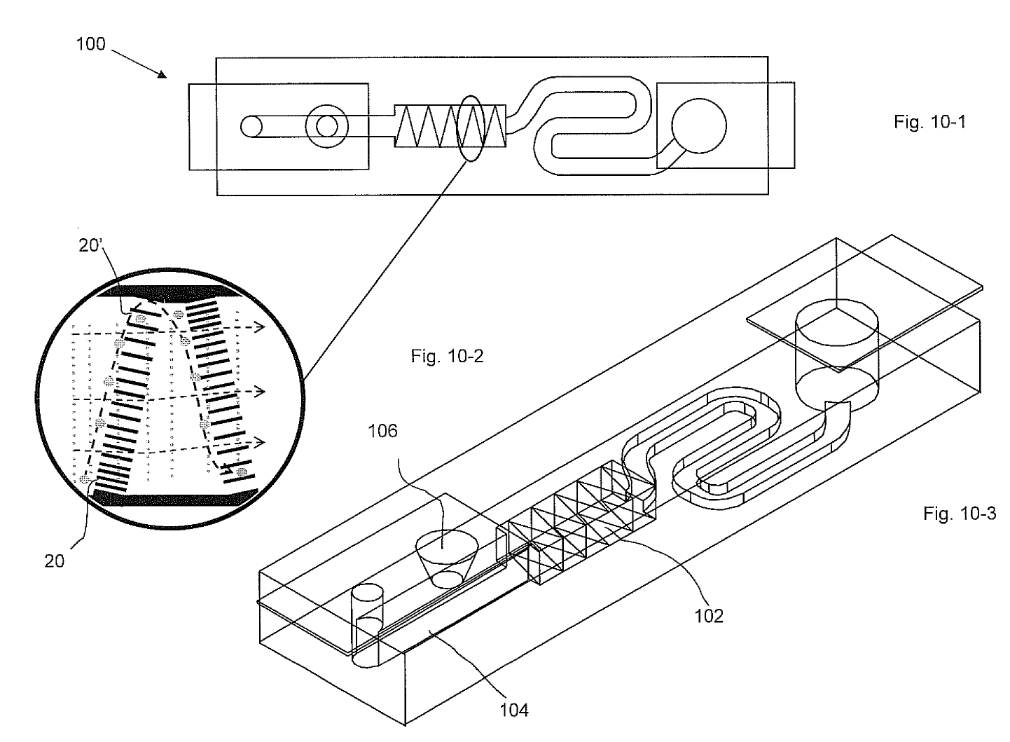

### DETAILED DESCRIPTION OF THE INVENTION

The present invention has utility to perform size exclusion based separation of particles dissolved or suspended in a liquid. The present invention is particularly well suited for qualitative and quantitative testing of parasite loading a liquid sample such as blood or water even in instances when the parasite is present as an infective in part per million levels based on the total number of erythrocytes.

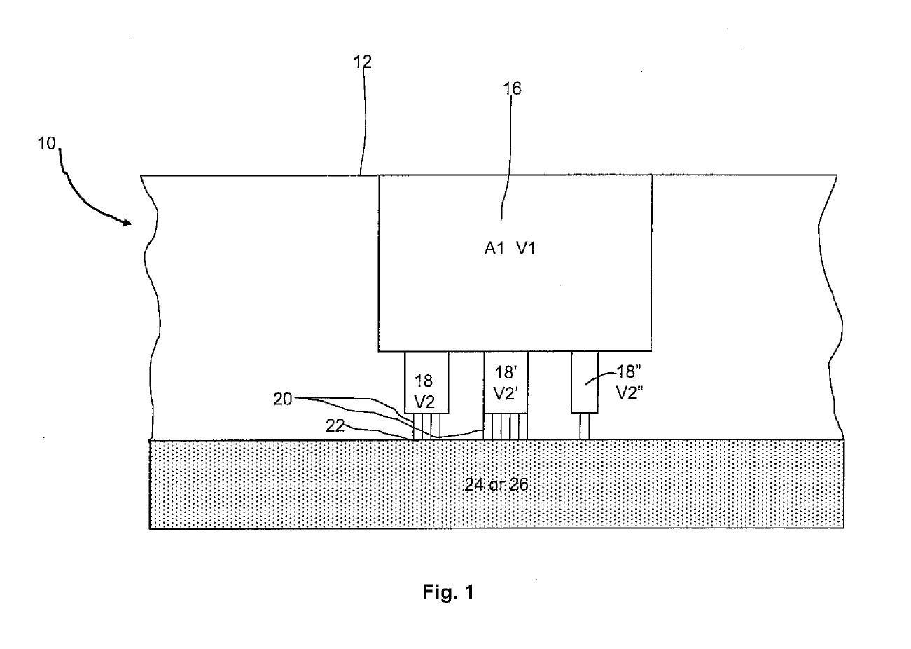

An inventive microseparation structure is shown in cross section in FIG. 1 and is characterized by three interconnected layers of dimensionally decreasing chambers and is shown generally at 10. A top surface 12 has a depression 14 therein having a cross-sectional area at surface 12 A1 and a volume V1 to define a first chamber 16. The chamber 16 is in fluid communication with multiple second chambers 18, 18′ and 18″ where each of the second chambers has a volume V2, V2′ and V2″, respectively, that is less than V1. As shown in FIG. 1, second chambers 18, 18′ and 18″ are optionally formed to have like volumes or different volumes relative to other second chambers. The second chambers 18-18″ are each interconnected with one or more particle size excluding holes 20. The distal end 22 of holes 20 is in fluid communication with a void 24 or a capillary draw agent 26 so as to induce capillary draw of liquid placed within first chamber 16 through second chambers 18-18″ and through holes 20 so as to retain size excluded particles of interest at the interface between chambers 18-18″ and holes 20. It is appreciated that the relative size of first chamber 16, secondary chambers 18-18″ and holes 20 are selected to facilitate selections as to aliquot size (first chamber 16), spatial concentration of separated particles (second chambers 18-18″) and the nature of the particle separated (holes 20).

An inventive microseparation structure 10 is readily formed using lithography techniques or hole formation in separate layers and then aligning and securing the layers together to define a structure 10. An attribute of an inventive structure 10 is that excess sampling liquid overflowing first chamber 16 is limited to an aliquot filling the volume V1 thereby obviating the need to pipette the specific sample volumes for separation. Additionally, with resort to capillary draw to move liquid within first chamber 16 through to the distal end 22 of holes 20, particle separation occurs with a controlled rate and pressure that is unlikely to damage or deform separated particles, all without resort to external laboratory equipment.

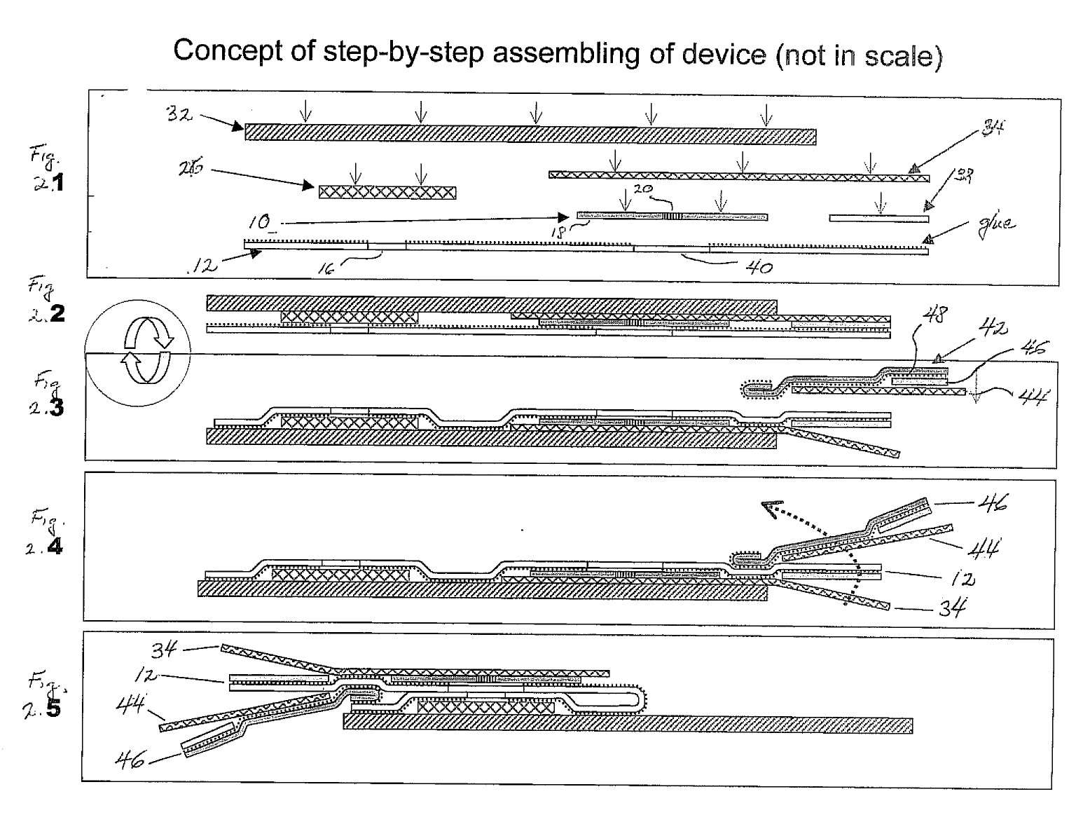

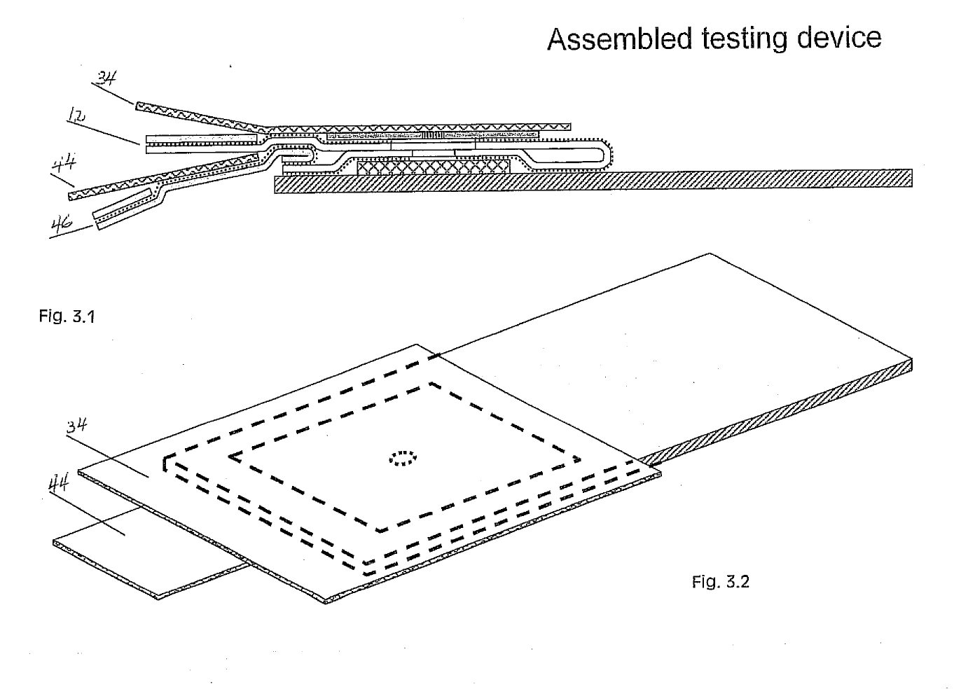

A filtration device for separating in particular a small number of particles from within a liquid using an inexpensive field suitable disposable device is detailed with respect to FIGS. 2.1-2.5 and FIGS. 3.1-3.4 generally at 30 where like numbers have the same meaning as used with respect to FIG. 1. The device 30 has holes 20 of a few microns in diameter and is placed on top of porous media such as filter paper 26. A drop of liquid such as blood is placed into first chamber 16 formed in a top surface 12 with the porous media 26 through capillary forces drawing the device 30 is constructed with liquid-impervious substrate 32 onto which media 26 is attached adjacent to a cover layer 34. The cover layer 34 is in turn overlayered with a portion containing at least one secondary chamber 18 defined by a secondary chamber volume V2 and terminating in holes 20. This separator sheet 36 is optionally provided with a spaced apart liquid impervious polymer sheet material 38 serving as a spacer and barrier and is also joined to cover layer 34. The separation sheet 36, optional spacer 38, and media 26 are in turn overlayered with a top surface 12 having a first chamber 16 and optionally a second aperture 40 therein. The assembled device 30 is shown in inverted form in FIG. 2.2. An optional blotter structure 42 is adhered to an edge of the device 30. The blotter structure 42 includes a liquid-impervious layer 44 formed of the same material as cover layer 32 and absorbent pad 46 with a blotter substrate layer 48. To render the device 30 in operational form, the cover layer 34 as well as the optional blotter 42 are partially pulled from substrate 32 and folded back to bring apertures 40 and 16 in the top layer 12 into registry, as shown sequentially in FIGS. 2.4 and 2.5.

The operation of the device 30 is sequentially depicted in FIGS. 3.1-3.4 that each show the device 30 in paired cross-sectional and perspective views where the cross-sectional view of FIG. 3.1 corresponds to FIG. 2.5, where like parts depicted in FIGS. 3.1-3.4 correspond to the meaning attributed to those with respect to FIGS. 2.1-2.5.

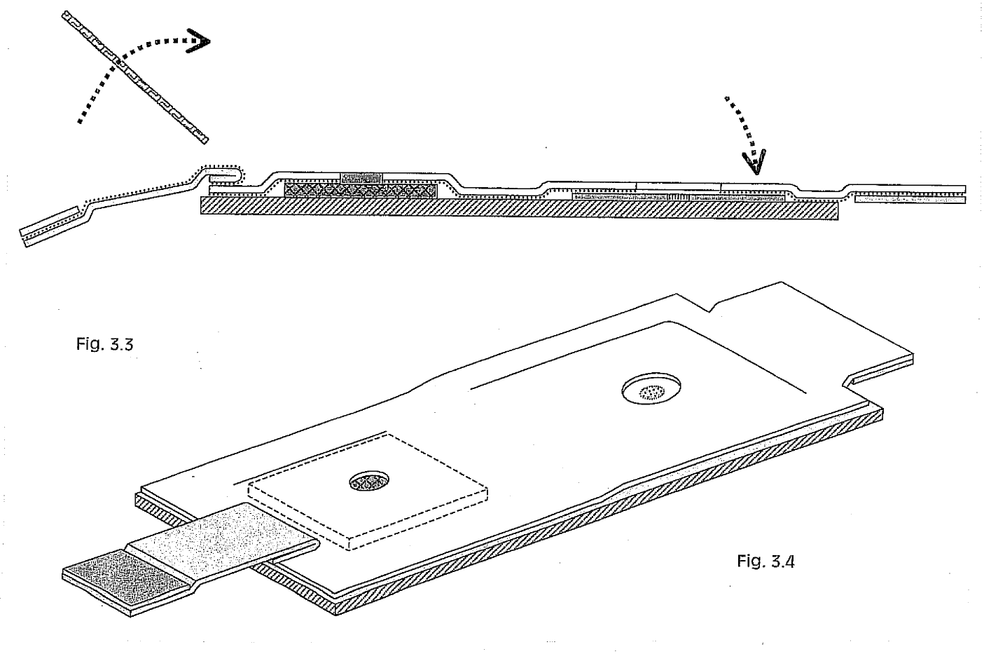

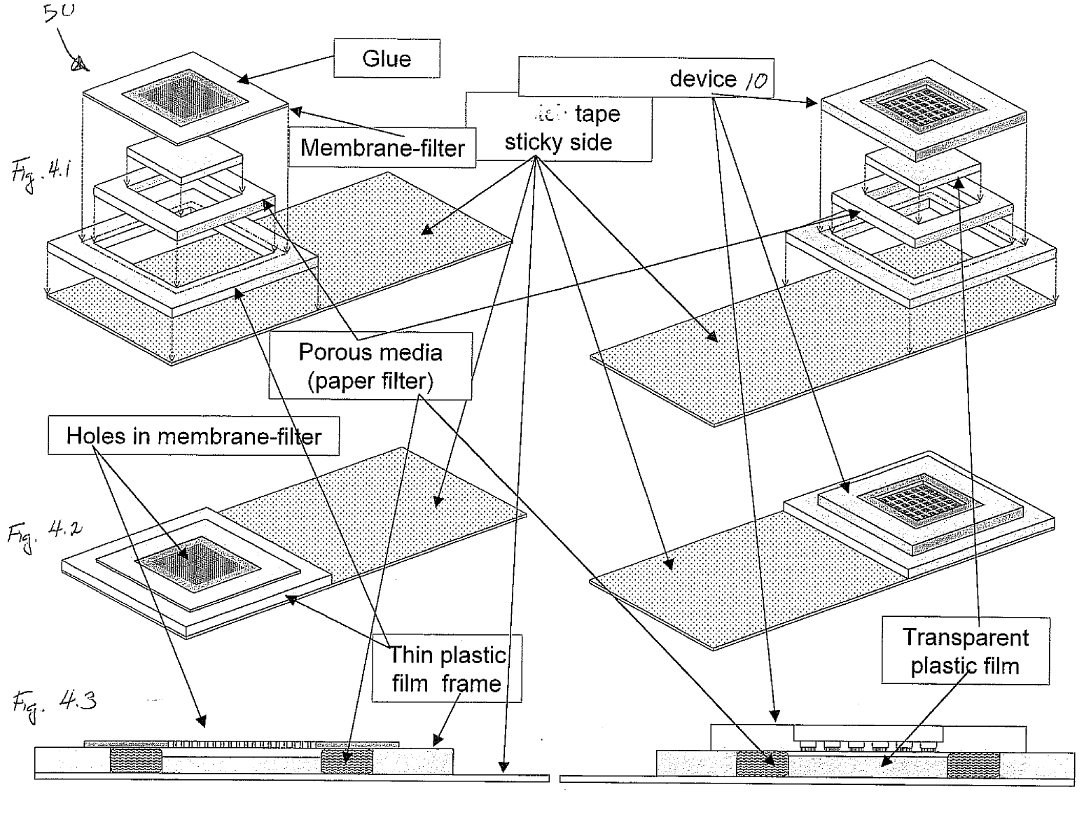

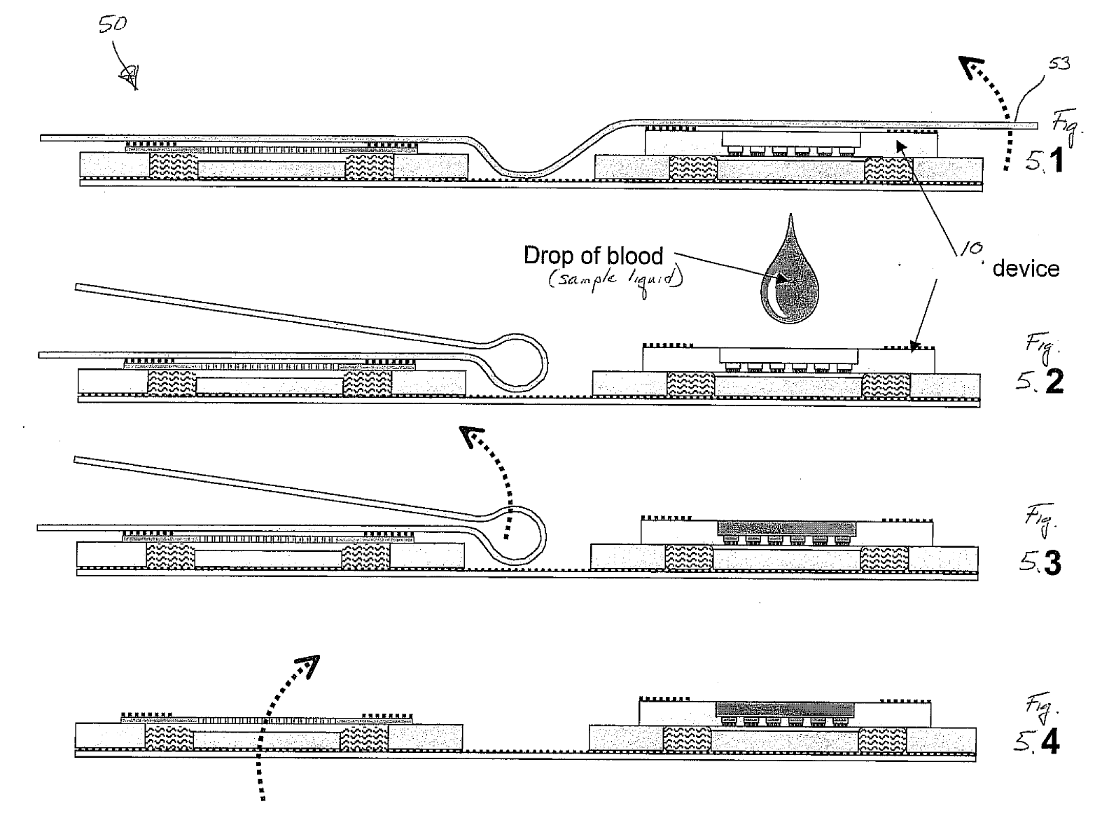

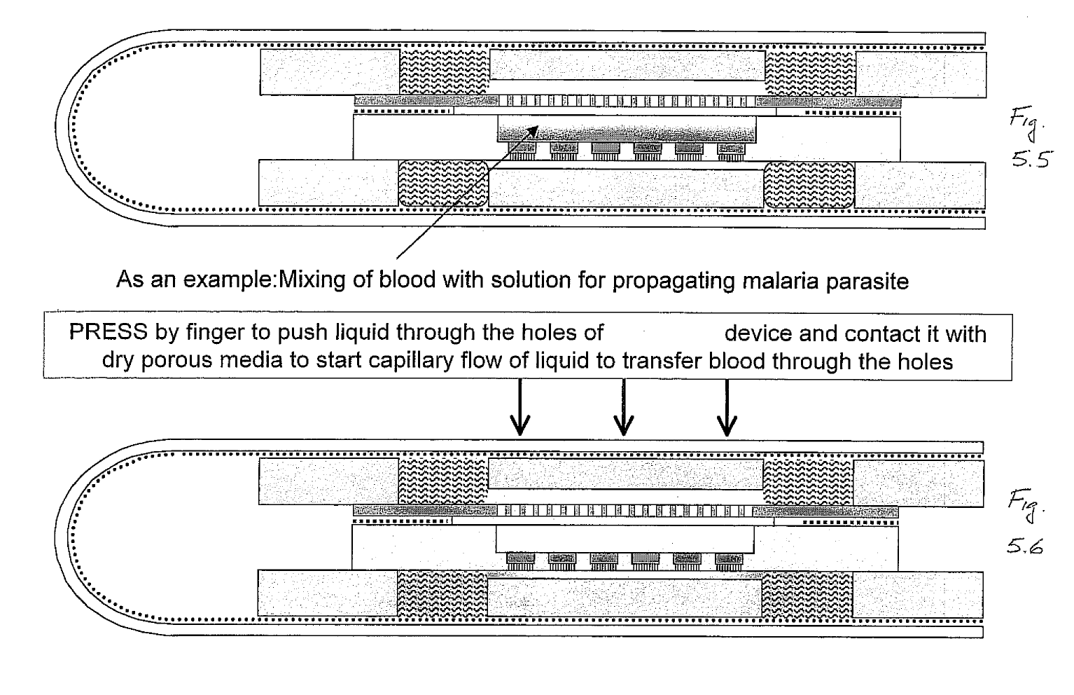

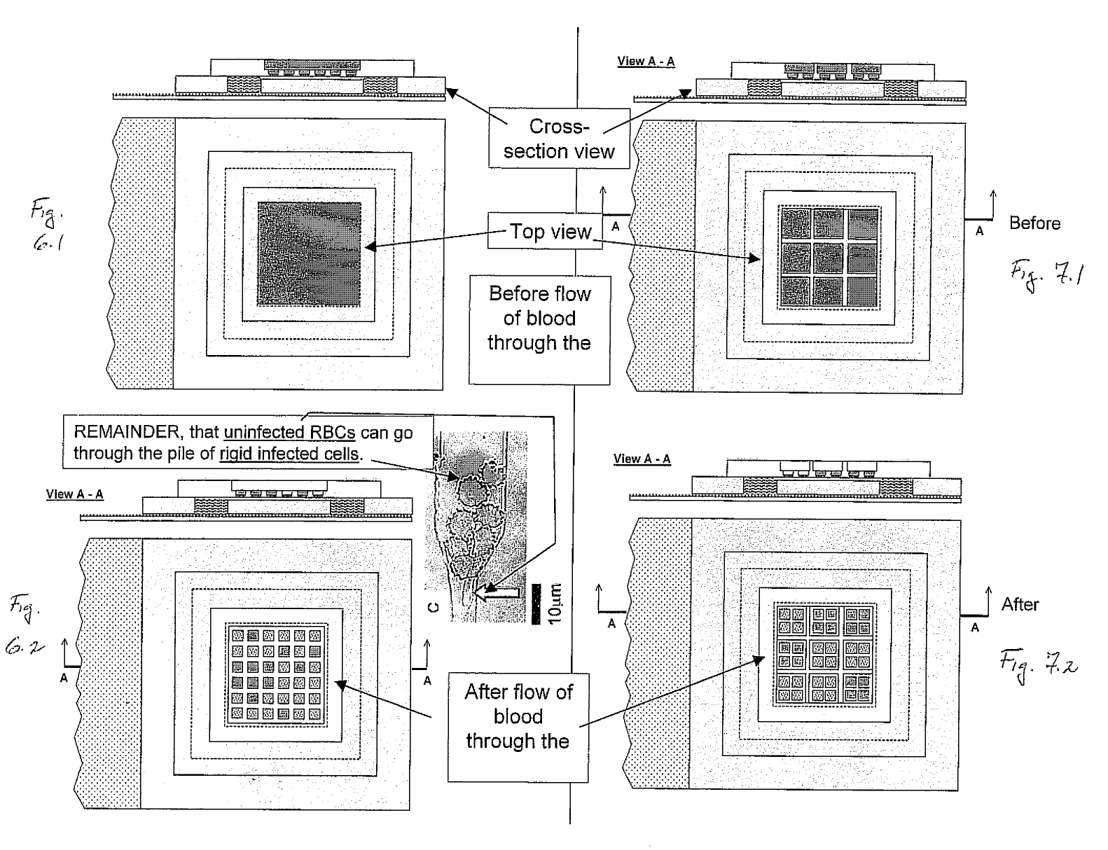

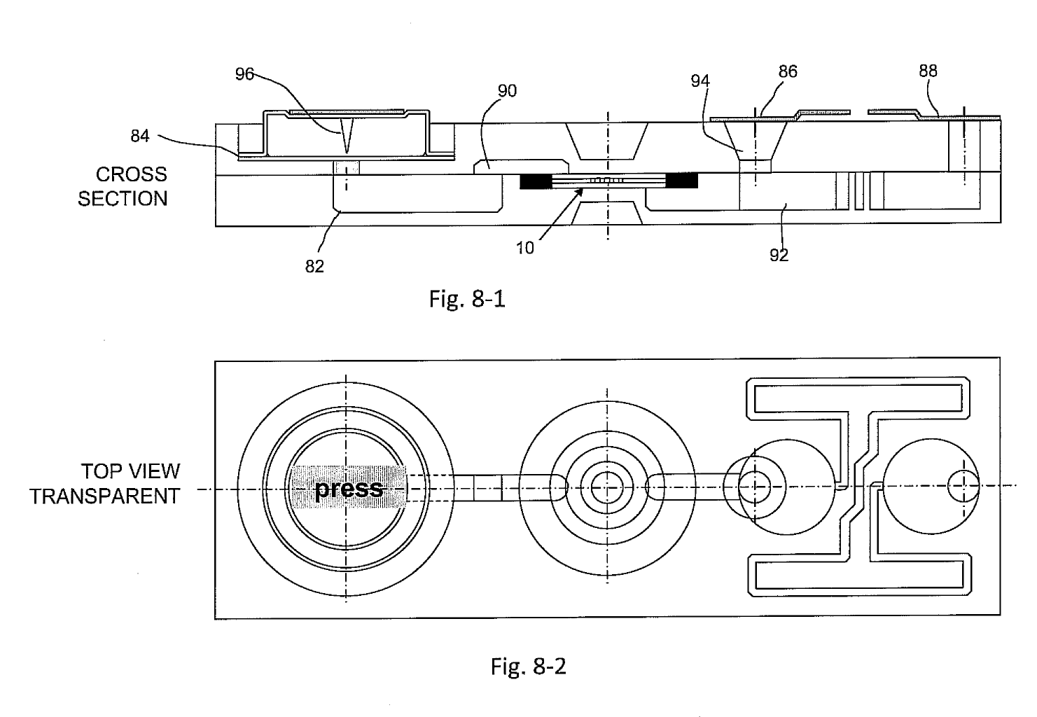

FIG. 4 shows a device 50 built around structure 10 detailed with respect to FIG. 1 that is placed between two pieces of glass or filter paper combinations thereof in which one sandwich layer is drywall and the other is saturated with a solution to be sampled. Liquid containing articles of interest flows through the holes in the membrane filter, through the structure 10, and is absorbed in the porous media filter frame surrounding structure 10. It is appreciated that the structure 10 is readily formed with a common first chamber 16 as best seen in FIG. 4C or alternatively that chamber 16 is divided into separate subchambers thereby allowing for parallel multiple sampling with each first chamber subchamber forming a sampling well. The operation of the device shown through construction images 4.1-4.3 is depicted sequentially in FIGS. 5.1-5.6 with the removal of a protective cover layer 53. The operation of such a device with a common first chamber in the exemplary case where the liquid sample is a blood sample potentially containing malaria infected blood cells is depicted in FIGS. 6.1 and 6.2 of the device 50. FIGS. 7.1 and 7.2 parallel those of FIGS. 6.1 and 6.2 with the exception that the first chamber has internal partitions to create first chamber subchambers allowing for parallel sampling. Owing to the size of rigid infected malaria cells as depicted in the insert between FIGS. 6.2 and 7.2, malaria infected blood cells remained trapped within the structure 10 while uninfected red blood cells pass therethrough leaving a red visual appearance in the top view where malaria infected red blood cells are trapped within the structure 10. Size exclusion of malaria infected red blood cells is known to the art as detailed in PNAS, Dec. 9, 2003, 100(25) 14618-14622. A microfluidics embodiment of an inventive device is depicted at 80 in FIGS. 8.1-8.3 inclusive of an inventive structure 10. It is appreciated that a device 80 built on a transparent substrate 82 makes the device amenable to subsequent investigation with an optical microscope. The device 80 has a channel 82 loaded with a carrier liquid such as saline solution, biological samples, or other inert buffer solutions during manufacture. Membrane seals 84, 86 and 88 retain the liquid within channel 82 prior to usage. The channel 82 is connected with the structure 10 through cavity 90. Cavity 92 bounds the other side of structure 10 and has a portal 94 for receiving a test liquid therein. A spike 96 is resiliently suspended above membrane 84 such that upon depression of spike 96, the membrane 84 can be ruptured. Operation of the device 80 is shown in FIGS. 9.1-9.4 with the test liquid being a drop of blood potentially containing malaria infected red blood cells which have poor deformation and are therefore caught in an inventive structure 10.

Another inventive device is depicted at 100 containing a tape made from a thin membrane filter with holes of various sizes of a few microns in diameter 102 inserted within the flow path of the channel 104. As a result, liquid that enters the channel 104 through inlet 106 traverses channel 104 and penetrates through the holes 20 of a first diameter or of a second diameter 20′ within membrane filter 102 thereby forcing flexible malaria infected blood cells to follow a greater path inside the channel than uninfected cells, thus allowing for the collection of two separate fractions of infected and uninfected red blood cells as a function of time from the reservoir 108.

Patent documents and publications mentioned in the specification are indicative of the levels of those skilled in the art to which the invention pertains. These documents and publications are incorporated herein by reference to the same extent as if each individual document or publication was specifically and individually incorporated herein by reference.

The foregoing description is illustrative of particular embodiments of the invention, but is not meant to be a limitation upon the practice thereof. The following claims, including all equivalents thereof, are intended to define the scope of the invention.

1. A microseparation structure comprising:
a top surface defining a first chamber having a first chamber area and a first chamber volume;
a second chamber in fluid communication with said first chamber, said second chamber having a second chamber volume less than the first chamber volume; and
at least one hole in fluid communication with said second chamber, said at least one hole having a distal end in fluid communication with a capillary draw void or a capillary draw inducing agent such that a liquid contacting said first chamber is drawn through said second chamber and the at least one hole and past the distal end to segregate particles within the liquid in said second chamber when the particles are unable to enter said at least one hole.

2. The structure of claim 1 wherein the distal end is in fluid communication with a capillary draw agent.

3. The structure of claim 2 wherein said capillary draw agent is a nonwoven fiber material, packed particulate, or porous material wet by the liquid.

4. The structure of claim 1 further comprising a plurality of second chambers each having a volume less than the first chamber volume.

5. A blood testing device comprising a plurality of microseparation structures of claim 1.

6. The device of claim 5 wherein said first chamber and said second chamber of a structure of claim 1 are foldably separable.

7. The device of claim 5 wherein the distal end of said at least one hole of the structure of claim 1 is compressed relative to the void or capillary draw agent to induce flow of a liquid therethrough.

8. The device of claim 5 wherein said plurality of structures of claim 1 vary in the volume of at least one of first chambers, second chambers, and cross-sectional area of the at least one hole between separate of the structures of claim 1.

9. A chromatographic filtration media comprising:
a plurality of microseparation structures of claim 1 formed in a folded sheet.
